(19) United States
(12) Patent Application Publication
(10) Pub. No.: US 2024/0207396 A1
(43) Pub. Date: Jun. 27, 2024

(54) OLIGOSACCHARIDE VACCINE FOR SPECIFIC PREVENTION OF FUNGAL INFECTIONS AND METHOD FOR PREPARING SAME

(71) Applicant: SHANDONG ACADEMY OF PHARMACEUTICAL SCIENCES, Shandong (CN)

(72) Inventors: Fei LIU, Shandong (CN); Dandan YUAN, Shandong (CN); lei CHEN, Shandong (CN); Linlin NIU, Shandong (CN); Mian CHEN, Shandong (CN); Huarong SHAO, Shandong (CN); Xiaogang ZHANG, Shandong (CN); Yingmei LIU, Shandong (CN); Jinhua ZHANG, Shandong (CN); Mengqi DIAO, Shandong (CN); Xinyan GUO, Shandong (CN)

(73) Assignee: SHANDONG ACADEMY OF PHARMACEUTICAL SCIENCES, Shandong (CN)

(21) Appl. No.: 18/557,597

(22) PCT Filed: Jul. 13, 2021
(86) PCT No.: PCT/CN2021/000153

§ 371 Date: Oct. 27, 2023

(30) Foreign Application Priority Data
Apr. 30, 2021 (CN) 202110484142.X

(51) Int. Cl. A61K 39/39; A61K 39/00; A61P 31/10; A61P 37/04

(52) U.S. Cl. CPC A61K 39/39; A61P 31/10; A61P 37/04; A61K 2039/57; A61K 2039/6087

(57) ABSTRACT

An oligosaccharide vaccine for specific prevention of fungal infections and a method for preparing the same are provided. The vaccine is formed by conjugating a sulfhydrylated protein with oligosaccharide. The sulfhydrylated protein is formed by introducing a sulfhydryl group (—SH) to a carrier protein containing a primary amino group (—NH2), and then is conjugated with the oligosaccharide to form the oligosaccharide vaccine under the binding action of a bridging agent. The carrier protein is a non-humanized protein, and the oligosaccharide is a chitosan oligosaccharide mixture and/or chitin oligosaccharide mixture. The vaccine has strong immunogenicity, can activate Th17 cell immunity, and can recognize and protect infections caused by fungi.

### BACKGROUND OF THE INVENTION

### 1. Technical Field

The present invention belongs to the field of biotechnology, and particularly relates to an oligosaccharide vaccine for specific prevention of fungal infections and a method for preparing the same.

### 2. Background Art

Information of the background is merely disclosed to increase the understanding of the overall background of the present invention, but is not necessarily regarded as acknowledging or suggesting, in any form, that the information has constituted the prior art known to a person of ordinary skill in the art.

Fungi are widespread microorganisms in nature and common bacteria that colonize on the surface of human skin and mucous membranes. However, when an immune system is compromised or a host barrier is broken, the fungi can invade a human body, leading to fatal infections. According to incomplete statistics, more than 400 out of 1.5 million species of fungi in this world can cause serious infection in humans. Most systemic mycoses are caused by Candida, Cryptococcus and Aspergillus. Compared with those of other microbial infections, clinical understanding of systemic fungal infections is not deep, diagnostic methods are limited, and many patients are not treated in time.

In recent years, although there are more and more anti-fungal drugs, the efficacies of the anti-fungal drugs are limited due to their limited antimicrobial spectra, toxic and side effects, fungal resistance and the like. Research shows that fungal vaccines have a good application prospect in aspect of development in comparison with conventional anti-fungal drugs. With a glycoprotein compound in which β-glucan, synthetic mannose and carrier protein are conjugated as a vaccine, the effect of preventing invasive fungal infections is achieved. However, since a complex chemical synthesis process is required for an antigen under test, the number of varieties that can be used for screening effective carbohydrate antigens is limited, and the protective action of vaccines under research at present still needs to be improved. In other research, sulfhydrylated chitosan oligosaccharide is linked with a heterobifunctional linker to form an adjuvant, and then the adjuvant is conjugated with an attenuated vaccine, a protein vaccine and the like. This chitosan oligosaccharide adjuvant can enhance the immune response, but since the chitosan oligosaccharide contains multiple amino groups, after being sulfhydrylated, a multi-site sulfhydrylated chitosan oligosaccharide is conjugated with multiple vaccines, which is equivalent to that multiple amino antigenic determinants of the chitosan oligosaccharide are modified, and the chitosan oligosaccharide is no longer a complete mechanism of constituents of a fungal cell wall. Therefore, the vaccines prepared therefrom cannot stimulate an immune response in which fungi are effectively recognized.

Chitin is a constituent that widely exists in the fungal cell wall. Chitosan oligosaccharide is a small-molecule oligosaccharide obtained from enzymatic hydrolysis after deacetylation of chitin. Chitin oligosaccharide is a small-molecule oligosaccharide obtained from the enzymatic hydrolysis of the chitin. All of them have a monosaccharide composition similar to the chitin. Thus, they can be used as a broad-spectrum antifungal target, and have certain immunogenicity. However, broad-spectrum fungal vaccines in this field have not yet been reported, and their mechanisms such as recognition by the immune system are still unclear. Therefore, the research on anti-fungal vaccines based on fungal cell structure has very urgent practical significance.

### SUMMARY OF THE INVENTION

In order to overcome the above problems, an objective of the present invention is to provide a vaccine for specific prevention of fungal infections, used for preventing and treating infections caused by fungi in immunosuppressed patients.

In order to realize the above technical objective, the present invention employs the following technical solution:

In a first aspect of the present invention, an oligosaccharide vaccine for specific prevention of fungal infections and a method for preparing the same are provided, the method including:
performing sulfhydrylation modification on a carrier protein containing a primary amino group to obtain a sulfhydrylated protein; and
conjugating the sulfhydrylated protein with a chitosan oligosaccharide mixture and/or chitin oligosaccharide mixture to form the oligosaccharide vaccine.

The research finds that: the oligosaccharide vaccine formed by conjugating the sulfhydrylated protein with the oligosaccharide has a clear conjugation site and strong specificity; and at the same time, the oligosaccharides employed are chitosan oligosaccharide mixtures and/or chitin oligosaccharide mixtures with different polymerization degrees, which are more likely to cause immune responses of cells. Therefore, a final oligosaccharide vaccine can better recognize and prevent infections caused by fungi (such as Candida albicans, Aspergillus, and Cryptococcus neoformans).

In a second aspect of the present invention, an oligosaccharide vaccine for specific prevention of fungal infections prepared by any of the above methods is provided.

In a third aspect of the present invention, application of the above oligosaccharide vaccine for specific prevention of fungal infections to preparation of drugs for treating or preventing systemic fungal infections, or drugs for activating Th17 cell immune responses.

The present invention has the following beneficial effects:
(1) The oligosaccharide vaccine provided by the present invention is obtained by introducing a sulfhydryl group (—SH) to the carrier protein containing the primary amino group (—NH₂) to form the sulfhydrylated protein, and then conjugating the sulfhydrylated protein with the oligosaccharide under the binding action of a bridging agent. The conjugation site of the oligosaccharide vaccine is clear. It is conjugated to a reducing end group (free aldehyde group, —CHO) of the oligosaccharide, maintains the original structural characteristics of the oligosaccharide, and has strong specificity. The chitosan oligosaccharide and/or chitin oligosaccharide employed are/is mixture(s) of different polymerization degrees. That is, by increasing the polymerization degree and the diversity of acetyl group distribution, the prepared vaccine has strong immunogenicity, can activate the Th17 cell immune response, and can recognize and prevent infections caused by fungi (such as C. albicans, Aspergillus, and C. neoformans).
(2) This application has a simple operation method, low costs, and universality, which is beneficial for mass production.

### DETAILED DESCRIPTION OF THE EMBODIMENTS

It should be noted that, the following detailed descriptions are all exemplary, and are intended to provide further descriptions of the present invention. Unless otherwise specified, all technical and scientific terms used in the present invention have the same meanings as those usually understood by a person of ordinary skill in the art to which the present invention belongs.

It should be noted that the terms used herein are merely used for describing specific implementations, and are not intended to limit exemplary implementations of the present invention. As used herein, the singular form is intended to include the plural form, unless the context clearly indicates otherwise. In addition, it should further be understood that terms “comprise” and/or “include” used in this specification indicate that there are features, steps, operations, devices, components, and/or combinations thereof.

### Interpretation of Terms

In this application, SATA refers to: N-succinimidyl-S-acetylthioacetate.

In this application, PDPH refers to: 3-(2-pyridinedithio) propionohydrazide.

An oligosaccharide vaccine for specific prevention of fungal infections is provided. The vaccine is a conjugate of an oligosaccharide and a sulfhydrylated protein. The sulfhydrylated protein is formed by introducing a sulfhydryl group (—SH) to a carrier protein containing a primary amino group (—NH₂), and then is conjugated with the oligosaccharide to form the oligosaccharide vaccine under the binding action of a bridging agent. The conjugation molar ratio of the oligosaccharide to the carrier protein is (200 to 500):1 or above.

The oligosaccharide is a chitosan oligosaccharide mixture and/or chitin oligosaccharide mixture, both of which are mixtures of different polymerization degrees, with a relative molecular weight of ≤5000 Da and a deacetylation degree of 0-100%.

The protein is a non-humanized protein, selected from one of Concholepasconcholepas hemocyanin (CCH), keyhole limpet hemocyanin (KLH), bovine serum albumin (BSA), tetanus toxin/toxoid, rotavirus VP7 protein, a diphtheria toxin mutant CRM, and Clostridium perfringens exotoxin/toxoid.

The method for preparing the sulfhydrylated protein includes:
(1) adding SATA into dimethylformamide (DMF) to prepare a 2-10 mg/ml SATA/DMF solution;
(2) preparing a 1-10 mg/ml protein solution from a phosphate buffer, adding the SATA/DMF solution with a molar amount that exceeds 5-100 times that of the protein solution, and performing reaction at room temperature for 20-80 min;
(3) adding hydroxylamine·HCl to the phosphate buffer to prepare a 10-100 mg/ml hydroxylamine solution; and
(4) adding a certain amount of hydroxylamine solution to the SATA-protein solution, and performing the reaction at room temperature for 0.5-4 h to obtain a sulfhydrylated protein solution.

In step (4) of the method for preparing the sulfhydrylated protein, the addition amount of the hydroxylamine solution is 100 μl of the hydroxylamine solution per milliliter of the protein solution.

The conjugation of the oligosaccharide with the sulfhydrylated protein includes:
(1) adding the bridging agent to dimethylformamide (DMF) to prepare a 5-100 mM bridging agent solution;
(2) adding the bridging agent with a molar amount that exceeds 5-50 times that of the sulfhydrylated protein into the sulfhydrylated protein solution prepared in step (4) of the method for preparing the sulfhydrylated protein, and performing the reaction at room temperature for 1-6 h;
(3) adding the oligosaccharide, and performing the reaction at 4° C. for 1-7 d; and
(4) removing small-molecule impurities to obtain the oligosaccharide vaccine.

In steps (1) (2) of conjugation of the sulfhydrylated protein with the oligosaccharide, the bridging agent is PDPH.

In step (3) of conjugation of the oligosaccharide with the sulfhydrylated protein, the molar concentration of the oligosaccharide is equivalent to 100-1200 times the molar amount of the sulfhydrylated protein.

In step (4) of conjugation of the oligosaccharide with the sulfhydrylated protein, the method for removing the small-molecule impurities is dialysis or column chromatography. The buffer used for the column chromatography or dialysis is a solution used for dissolving the carrier protein. The packing model of a desalting column is one of Sephadex G10-G50, Bio-gel P2-P10 and Thermo Scientific™Zeba™ Spin Desalting Columns. A dialysis bag for a dialysis method has a molecular weight cut-off of 1000-10000 Da, and a flat width of 1-3.5 cm.

The phosphate buffer mentioned above is 0.01-0.1 M sodium phosphate, and 0-0.15 M sodium chloride, with pH of 7.0 to 8.0.

The above oligosaccharide vaccine is used for treating or preventing infections caused by systemic fungi, including, but not limited to, C. albicans, Aspergillus and C. neoformans.

The present invention is further described below in conjunction with specific examples. It should be noted that the specific examples are an explanation of the present invention rather than a limitation.

### EXAMPLE 1

### (1) Preparation of Sulfhydrylated Protein

SATA was added into DMF to prepare a 2 mg/ml SATA/DMF solution. 2 ml of a 2 mg/ml carrier protein (BSA) solution was prepared from a 0.01 M sodium phosphate buffer (pH 7.0). The SATA/DMF solution with a molar amount that exceeds 5 times that of the carrier protein was added. The reaction was performed at room temperature for 30 min. Hydroxylamine·HCl was added into a 0.01 M sodium phosphate buffer (pH 7.0) to prepare a 20 mg/ml hydroxylamine solution. 200 μL of the hydroxylamine solution was added into the SATA-protein solution. The reaction was performed at room temperature for 2 h to obtain a sulfhydrylated protein solution.

### (2) Conjugation of Oligosaccharide with Sulfhydrylated Protein

PDPH was added into DMF to prepare a 5 mM PDPH/DMF solution. PDPH with a molar amount that exceeds 5 times that of the sulfhydrylated protein was added into the sulfhydrylated protein solution prepared in (1) mentioned above. The reaction was performed at room temperature for 1 h. Chitosan oligosaccharide with a deacetylation degree of 85% and a molecular weight of ≤5000 Da, which was equivalent to 100 times the molar amount of the protein, was added. The reaction was performed at 4° C. for 1 d. With 0.01 M sodium chloride (pH 7.0) as a buffer for dialysis, small-molecule impurities were removed by a dialysis bag with a molecular weight cut-off of 5000 Da to obtain a chitosan oligosaccharide-carrier protein conjugate. Vacuum freeze drying was performed to obtain the oligosaccharide vaccine.

### EXAMPLE 2

### (1) Preparation of Sulfhydrylated Protein

SATA was added into DMF to prepare a 3 mg/ml SATA/DMF solution. 3 ml of a 4 mg/ml carrier protein (CCH) solution was prepared from a 0.01 M sodium phosphate and 0.01 M sodium chloride buffer (pH 7.2). The SATA/DMF solution with a molar amount that exceeds 80 times that of the carrier protein was added. The reaction was performed at room temperature for 50 min. With 0.01 M sodium phosphate and 0.01 M sodium chloride (pH 7.2) as a buffer for dialysis. Small-molecule impurities were removed by a dialysis bag with a molecular weight cut-off of 10000 Da. The dialysate was collected to obtain a SATA-protein solution. Hydroxylamine·HCl was added into the 0.01 M sodium phosphate and 0.01 M sodium chloride buffer (pH 7.2) to prepare a 40 mg/ml hydroxylamine solution. A certain amount of hydroxylamine solution was added into the SATA-protein solution. The reaction was performed at room temperature for 1 h to obtain a sulfhydrylated protein solution.

### (2) Conjugation of Oligosaccharide with Sulfhydrylated Protein

PDPH was added into DMF to prepare a 20 mM PDPH/DMF solution. PDPH with a molar amount that exceeds 15 times that of the sulfhydrylated protein was added into the sulfhydrylated protein solution prepared in (1) mentioned above. The reaction was performed at room temperature for 1 h. Dialysis was performed. The dialysate was collected. Chitin oligosaccharide with a deacetylation degree of 5% and a molecular weight of ≤2000 Da, which was equivalent to 500 times the molar amount of the protein, was added. The reaction was performed at 4° C. for 2 d. Small-molecule impurities were removed by dialysis to obtain a chitin oligosaccharide-carrier protein conjugate. Vacuum freeze drying was performed to obtain the oligosaccharide vaccine.

### EXAMPLE 3

### (1) Preparation of Sulfhydrylated Protein

SATA was added into DMF to prepare a 5 mg/ml SATA/DMF solution. 2 ml of a 6 mg/ml carrier protein (KLH) was prepared with a 0.1 M sodium phosphate and 0.01 M sodium chloride buffer (pH 7.2). The SATA/DMF solution with a molar amount that exceeds 100 times that of the carrier protein was added. The reaction was performed at room temperature for 50 min. Hydroxylamine·HCl was added into the 0.1 M sodium phosphate and 0.01 M sodium chloride buffer (pH 7.2) to prepare a 100 mg/ml hydroxylamine solution. 200 μL of the hydroxylamine solution was added into the SATA-protein solution. The reaction was performed at room temperature for 3 h to obtain a sulfhydrylated protein solution.

### (2) Conjugation of Oligosaccharide with Sulfhydrylated Protein

PDPH was added into DMF to prepare a 30 mM PDPH/DMF solution. PDPH with a molar amount that exceeds 30 times that of the sulfhydrylated protein was added into the sulfhydrylated protein solution prepared in (1) mentioned above. The reaction was performed at room temperature for 3 h. Chitosan oligosaccharide with a deacetylation degree of 90% and a molecular weight of ≤2000 Da, which was equivalent to 800 times the molar amount of the protein, was added. The reaction was performed at 4° C. for 3 d. With a 0.1 M sodium phosphate and 0.01 M sodium chloride buffer (pH 7.2) as a mobile phase, through Bio-gel P2 Desalting Columns, after a sample flows through the column, the fractions were collected, with 10 drops (about 0.5 ml) per tube, and the sample of fraction was received for 20 consecutive tubes. After detection, a chitosan oligosaccharide-carrier protein conjugate was obtained. Vacuum freeze drying was performed to obtain the oligosaccharide vaccine.

### EXAMPLE 4

### (1) Preparation of Sulfhydrylated Protein

SATA was added into DMF to prepare a 6 mg/ml SATA/DMF solution. 2 ml of a 8 mg/ml carrier protein (tetanus toxin) was prepared from a 0.1 M sodium phosphate and 0.01 M sodium chloride buffer (pH 7.2). The SATA/DMF solution with a molar amount that exceeds 30 times that of the carrier protein. The reaction was performed at room temperature for 30 min. Hydroxylamine·HCl was added into the 0.1 M sodium phosphate and 0.01 M sodium chloride buffer (pH 7.2) to prepare a 100 mg/ml hydroxylamine solution. 200 μL of the hydroxylamine solution was added into the SATA-protein solution. The reaction was performed at room temperature for 3 h to obtain a sulfhydrylated protein solution.

### (2) Conjugation of Oligosaccharide with Sulfhydrylated Protein

PDPH was added into DMF to prepare a 50 mM PDPH/DMF solution. PDPH with a molar amount that exceeds 30 times that of the sulfhydrylated protein was added into the sulfhydrylated protein solution prepared in (1) mentioned above. The reaction was performed at room temperature for 3 h. Chitin oligosaccharide with a deacetylation degree of 2% and a molecular weight of ≤1000 Da, which was equivalent to 200 times the molar amount of the protein, was added. The reaction was performed at 4° C. for 4 d. With a 0.1 M sodium phosphate and 0.01 M sodium chloride buffer (pH 7.2) as a mobile phase, through desalting Bio-gel P2, after a sample flows through the column, the fractions were collected, with 10 drops (about 0.5 ml) per tube, and the sample of fraction was continuously received for 20 tubes. After detection, a chitin oligosaccharide-carrier protein conjugate was obtained. Vacuum freeze drying was performed to obtain the oligosaccharide vaccine.

### EXAMPLE 5

### (1) Preparation of Sulfhydrylated Protein

SATA was added into DMF to prepare a 5 mg/ml SATA/DMF solution. 2 ml of a 10 mg/ml carrier protein (diphtheria toxin mutant CRM) was prepared from a 0.1 M sodium phosphate and 0.15 M sodium chloride buffer (pH 8.0). The SATA/DMF solution with a molar amount that exceeds 15 times that of the carrier protein was added. The reaction was performed at room temperature for 30 min. Hydroxylamine·HCl was added into the 0.1 M sodium phosphate and 0.15 M sodium chloride buffer (pH 8.0) to prepare a 60 mg/ml hydroxylamine solution. 200 μL of the hydroxylamine solution was added into the SATA-protein solution. The reaction was performed at room temperature for 2 h to obtain a sulfhydrylated protein solution.

### (2) Conjugation of Oligosaccharide with Sulfhydrylated Protein

PDPH was added into DMF to prepare a 40 mM PDPH/DMF solution. PDPH with a molar amount that exceeds 20 times that of the sulfhydrylated protein was added into the sulfhydrylated protein solution prepared in (1) mentioned above. The reaction was performed at room temperature for 4 h. Chitosan oligosaccharide with a deacetylation degree of 95% and a molecular weight of ≤1000-3000 Da, which was equivalent to 200 times the molar amount of the protein, was added. The reaction was performed at 4° C. for 3 d. With a 0.1 M sodium phosphate and 0.15 M sodium chloride buffer (pH 8.0) as a mobile phase, through a desalting column Thermo Scientific™Zeba™ Spin, after a sample flows through the column, the fractions were collected, with 10 drops (about 0.5 ml) per tube, and the sample of fraction was continuously received for 20 tubes. After detection, a chitosan oligosaccharide-carrier protein conjugate was obtained. Vacuum freeze drying was performed to obtain the oligosaccharide vaccine.

### EXAMPLE 6

### (1) Preparation of Sulfhydrylated Protein

SATA was added into DMF to prepare a 10 mg/ml SATA/DMF solution. 2 ml of a 6 mg/ml carrier protein (KLH) was prepared from a 0.1 M sodium phosphate and 0.15 M sodium chloride buffer (pH 7.2). The SATA/DMF solution with a molar amount that exceeds 70 times that of the carrier protein was added. The reaction was performed at room temperature for 30 min. Hydroxylamine·HCl was added into the 0.1 M sodium phosphate and 0.15 M sodium chloride buffer (pH 7.2) to prepare a 100 mg/ml hydroxylamine solution. 200 μL of the hydroxylamine solution was added into the SATA-protein solution. The reaction was performed at room temperature for 3 h to obtain a sulfhydrylated protein solution.

### (2) Conjugation of Oligosaccharide with Sulfhydrylated Protein

PDPH was added into DMF to prepare an 80 mM PDPH/DMF solution. PDPH with a molar amount that exceeds 50 times that of the sulfhydrylated protein was added into the sulfhydrylated protein solution prepared in (1) mentioned above. The reaction was performed at room temperature for 5 h. Chitosan oligosaccharide with a deacetylation degree of 98% and a molecular weight of ≤3000 Da, which was equivalent to 900 times the molar amount of the protein, was added. The reaction was performed at 4° C. for 4 d. With a 0.1 M sodium phosphate and 0.15 M sodium chloride buffer (pH 7.2) as a mobile phase and a buffer for dialysis, small-molecule impurities were removed by a dialysis bag with a molecular weight cut-off of 10000 Da to obtain a chitosan oligosaccharide-carrier protein conjugate. Vacuum freeze drying was performed to obtain the oligosaccharide vaccine.

### EXAMPLE 7

### Determination of Conjugation Degree of Oligosaccharide-Sulfhydrylated Protein

Taking Example 6 as an example, the conjugation ratio of a chromatographic fraction sample was detected. The conjugation ratio was the mole number of the chitosan oligosaccharide attached onto each mole of carrier protein.

### (1) Determination of Reducing Sugar (Free Chitosan Oligosaccharide) Content in Sample

1 g of glucosamine hydrochloride (accurate to 0.001 g) was weighed, and dissolved in a ml volumetric flask with purified water. The solution was added to the scale line and then well shaken to obtain a 100 mg/ml standard solution mother solution. The standard solution mother solution was stored away from light in a refrigerator at 4° C. The standard solution mother solution was effective within two weeks. 0, 5, 10, 20, 30 and 40 μL of glucosamine standard solutions were each pipetted into a 1.5 ml centrifuge tube, and replenished to 1 ml with purified water to obtain different concentrations of standard solutions. At the same time, 5, 10 and 20 mg/ml chitosan oligosaccharide solutions were prepared with purified water.

30 μL of standard solution or samples to be determined was pipetted. 30 μL of DNS was added. Well mixing was performed. The mixture was treated in a boiling water bath for 5 min, and cooled with tap water. 180 μL of purified water was added, and well mixing was performed. 200 μL of mixture was taken and added into a 96-well plate. Absorbance OD₅₄₀ was determined with a microplate reader. With the molar concentration of the glucosamine hydrochloride as the abscissa and the absorbance as the ordinate, a standard curve was formulated, and the standard curve y=58.031x−0.0167, R²=0.9981 was obtained. The concentration of free chitosan oligosaccharide in the sample and chitosan oligosaccharide solutions was determined by the standard curve.

### (2) Determination of Total Sugar Content in Sample

Taking 100 mg/ml glucosamine hydrochloride in (1) as the standard solution mother solution, 0, 10, 30, 60, 90 and 120 μL of glucosamine standard solutions were each pipetted into a 1.5 ml centrifuge tube, and replenished to 1 ml with purified water to obtain different concentrations of standard solutions. At the same time, 5, 10 and 20 mg/ml chitosan oligosaccharide solutions were prepared with purified water.

0.1 ml of sample or standard solution was taken, and 0.3 ml of anthrone-sulfuric acid solution was added. The mixture was treated in a boiling water bath for 10 min, and immediately put in ice water and treated for 15 min. 200 μL of mixture was taken and placed into a 96-well plate. Absorbance OD₆₂₀ was determined with a microplate reader. With the molar concentration of the glucosamine hydrochloride as the abscissa and the absorbance as the ordinate, a standard curve was formulated, and the standard curve y=20.198x−0.017, R²=0.9977 was obtained. The concentration of total sugar in the sample and chitosan oligosaccharide solutions was determined by the standard curve.

### (3) Determination of Protein Content by Bradford Method

A 5 mg/ml BSA standard solution mother solution was prepared, and 0, 0.02, 0.05, 0.1, 0.3, 0.5 and 0.7 mg/ml standard solutions were prepared.

20 μL of standard solution or 10-fold diluted sample was taken and added into a 96-well plate. 200 μL of Coomassie brilliant blue 1×G250 staining solution was added into each well. The mixture was kept at room temperature for 3-5 min. Absorbance OD₅₉₅ was determined with a microplate reader. With the concentration of BSA as the abscissa and the absorbance as the ordinate, a standard curve was formulated, and the standard curve y=2.1838x+0.0294, R²=0.9948 was obtained. The concentration of protein in the sample was determined by the standard curve.

### (4) Calculation of Conjugation Ratio

Polymerization degree of chitosan oligosaccharide=mole number of total sugar of chitosan oligosaccharide/mole number of reducing sugar of chitosan oligosaccharide

Mole number mmol/ml of chitosan oligosaccharide attached to protein=(total sugar-reducing sugar×polymerization degree)/polymerization degree

Conjugation ratio=mole number of chitosan oligosaccharide attached to protein/(10×mole number of protein)

### (5) Result Analysis

By reducing sugar determination, it was found that there was no color reaction after dialysis, indicating that there was no reducing sugar (free chitosan oligosaccharide). The total sugar determination showed that the sample after dialysis showed brown and had a relatively large absorbance value, indicating that there was sugar. The protein was determined with the Bradford method. After dialysis, the sample had an obvious color reaction, showed bright blue, and had a relatively large absorbance value. The above results indicate that chitosan oligosaccharides were conjugated with the protein. According to determination data, the polymerization degree of the chitosan oligosaccharides is as shown in Table 1, and the conjugation ratio of the sample was calculated as 2395, and the data are as shown in Table 2.

TABLE 1
Polymerization Degree of Chitosan Oligosaccharide

| | | | Reducing | | |
|---|---|---|---|---|---|
| | Chitosan | Total sugar | sugar | | |
| Parallel | oligosaccharide | absorbance | absorbance | Polymerization | Polymerization |
| samples | concentration/mg · ml⁻¹ | OD₆₂₀ | OD₅₄₀ | degree | degree mean |
| Control | Water | 0.1353 | 0.0496 | / | / |
| 1 | 20 | 2.2896 | / | 4.17 | 5.18 |
| | 20 | / | 1.5288 | | |
| 2 | 10 | 1.3692 | / | 4.63 | |
| | 10 | / | 0.8081 | | |
| 3 | 5 | 1.0425 | / | 6.72 | |
| | 5 | / | 0.4276 | | |

TABLE 2
Conjugation Data of Chitosan Oligosaccharide and Carrier Protein

| Chromato- | Reducing | Total | Protein | |
|---|---|---|---|---|
| graphic | sugar | sugar | absorbance | Conjuga- |
| fraction | absorbance | absorbance | OD₅₉₅ after 10- | tion |
| sample | OD₅₄₀ | OD₅₄₀ | fold dilution | ratio |
| Control | 0.0493 | 0.1353 | 0.7649 | / |
| After dialysis | 0.0513 | 0.197 | 1.3922 | 2395 |

### EXAMPLE 8

### Immunological Experiment of Oligosaccharide Vaccine

The effectiveness of the vaccine was studied by using an immunocompromised (neutropenia) mouse model. This neutropenia mouse model was considered a suitable model, which can be used for simulating patients developing such deficiency due to treatment of various malignant tumors, chemotherapy and the like. Immune impairment induced by cyclophosphamide injection in mice would increase the sensitivity to fungi, which was similar to the increased susceptibility of neutropenia patients to the disease. To investigate the T cell-mediated immune response, mouse blood was taken and detected for a change in cytokine IL-17F level in Th17 cells to evaluate the immunogenicity of chitosan oligosaccharide-carrier protein vaccine.

### 1. Establishment of Immunocompromised Mouse Model

6-8-week-old Balb/c female mice were selected, and adaptively fed for 3-7 d. 20 g/L cyclophosphamide was prepared with normal saline, and the cyclophosphamide was injected intraperitoneally by body weight of 200 mg/kg. The number of neutrophils in the mouse blood was measured by a SYSMEX XT-2000i automatic blood analyzer after 3 days. If the number of the neutrophils was <500/mm³, the experiment could be continued. After that, the cyclophosphamide was injected every 10 days at a dose of 150 mg/kg, which was maintained throughout the test period.

### 2. Mouse Immunological Experiment Protocol

A normal saline blank control group, a KLH group, a chitosan oligosaccharide group, a chitosan oligosaccharide-KLH group in Example 6, a fluconazole group (as a positive control of a challenge test) were set up, with 10 mice in each group. At weeks 0, 2, 4 and 6 at the start of the experiment, the mice were injected subcutaneously from multiple points on the neck and back, with an injection amount of 2.5 mg/kg. The normal saline blank group was injected with normal saline, and the fluconazole group was not injected. The mice were intragastrically administrated by 50 mg/kg d at week 6 for 7 consecutive days. The other groups were injected with tested samples containing adjuvants.

In order to stimulate the organism to produce a strong immune response, Freund's adjuvants were selected. A Freund's complete adjuvant was selected for immunization at week 0, and a Freund's incomplete adjuvant was selected for immunization at each of weeks 2, 4 and 6. A 1 mg/mL solution was prepared from KLH, chitosan oligosaccharide, and chitosan oligosaccharide-KLH with a 0.02 M PBS buffer (pH 7.2-7.4). Then the solution was mixed with the Freund's complete adjuvant or Freund's incomplete adjuvant in an equal volume. The mixture was emulsified uniformly by ultrasonic waves.

### 3. Cytokine Detection

### Cytokine Determination Step:

(1) Extraction and stimulation of lymphocytes: Before the immunization at week 0 and after the immunization at week 6, 0.3 mL of blood was taken from the submandibular vein of the mouse and well mixed in a 1.5 mL anticoagulant tube. 3 mL of 1×red blood cell lysis buffer was added, and well mixed and reaction was performed for 4 min. Centrifugation was performed at 500 g at 4° C. for 10 min. The supernatant was sucked and discarded. Then, 10 mL of 0.01 M PBS was added, and then centrifugation was performed at 500 g for 10 min. The supernatant was sucked and discarded. Precipitated lymphocytes were suspended in a 96-well plate with 200 μL of stimulating solution, and incubated in a cell incubator for 4 h.
(2) Extracellular staining: The 96-well plate was centrifuged at 1600 rpm for 10 min (the same below as for centrifugal conditions). The supernatant was discarded. An FACS buffer containing extracellular staining antibodies (containing fluorescent stained CD3e, CCR6, CD4, and 7-AAD antibodies, with addition amounts of 1:100, 1:100, 1:200, and 1:100, respectively) was added by 70 μL/well and pipetted, and incubated away from light for 40 min, and extracellular staining was performed.
(3) Permeabilization: An FACS buffer without antibodies was added by 180 μL/well and pipetted. The supernatant was centrifuged and discarded. A permeabilization buffer was added by 100 μL/well, and incubated away from light at 4° C. for 40 min, and permeabilization treatment was performed.
(4) Intracellular staining: 1× permeabilization buffer was pipetted by 180 μL/well. The supernatant was centrifuged and discarded. A permeabilization buffer containing intracellular staining antibodies was added by 70 μL/well, and incubated away from light at room temperature for 40 min, and intracellular cytokine staining was performed.
(5) Cytokine detection: An FACS buffer was added by 180 μL/well and pipetted. The supernatant was centrifuged and discarded, and then the FACS buffer was added by 100 μL/well and pipetted. The intracellular IL-17F level of Th17 was detected by flow cytometry to reflect the activation of the tested sample on mouse cell immunity. The proportion of intracellular IL-17F to Th17 of the mice in each group before and after immunization is as shown in Table 3.

TABLE 3
Proportion of Intracellular IL-17F to Th17 before and after
Immunization (x̅ ± s, n = 10)

| | Before | After |
|---|---|---|
| Group | immunization/% | immunization/% |
| Blank group | 1.665 ± 0.332 | 1.289 ± 0.273 |
| KLH group | 1.456 ± 0.361 | 1.486 ± 0.355 |
| Chitosan oligosaccharide group | 1.511 ± 0.209 | 1.599 ± 0.329 |
| Chitosan oligosaccharide-KLH | 1.488 ± 0.337 | 1.937 ± 0.412**^{##} |
| group (Example 6) | | |

Note:
**P < 0.01 vs. before immunization,
^{##}P < 0.01 vs. blank group

From Table 3, it can be seen that compared with that before immunization, the proportion of intracellular IL-17F to Th17 of mice after immunization in the KLH group and the chitosan oligosaccharide group was increased, but the effect was not significant. The proportion of intracellular IL-17F of the mice in the chitosan oligosaccharide-KLH group in Example 6 was significantly increased (P<0.01). Compared with that of the blank control group, the proportion of intracellular IL-17F in the KLH group and the chitosan oligosaccharide group was increased to a certain extent. The proportion of intracellular IL-17F of the mice in the chitosan oligosaccharide-KLH group in Example 6 was significantly increased (P<0.01), indicating that oligosaccharide conjugated with the carrier protein could stimulate Th17 cell immunity, its secretion of cytokines IL-17F was promoted, and an anti-fungal role was played.

### EXAMPLE 9

### Challenge Test of Immunized Mice

The protective action of the oligosaccharide vaccine after a fungal challenge was studied by taking C. albicans as a challenge strain.

### 1. Determination of Fatal Dose by C. albicans Infection

The cultured C. albicans was made into a suspension with 0.01 M PBS, and the concentration was adjusted to 0.5×10⁶, 1.0×10⁶, 0.5×10⁷, 1.0×10⁷, 0.5×10⁸, 1.0×10⁸ CFU/ml. The mice were injected through the tail vein by a dose of 0.1 ml/mouse for challenge. The mice were selected from the immunocompromised mice (the number of neutrophils<500/mm³) injected with the cyclophosphamide in step 1 of Example 8. At the same time, a normal saline control group was set up, the death rate of the mice within 5 days was counted, and the least fatal dose was determined. The results are shown in Table 4.

TABLE 4
Fatality Rate by Tail Vein Injection of C. albicans into Mice

| | | | | | | | | Total | |
|---|---|---|---|---|---|---|---|---|---|
| Challenge | Number | Number of deaths at | | | | | | number of | Death |
| dose/CFU | of | different time | | | | | | deaths | rate/ |
| per mouse | mice | 12 h | 1 d | 2 d | 3 d | 4 d | 5 d | in 5 days | % |
| Control | 20 | 0 | 0 | 0 | 0 | 0 | 0 | 0 | 0 |
| group | | | | | | | | | |
| 0.5 × 10⁵ | 20 | 0 | 0 | 0 | 0 | 0 | 0 | 0 | 0 |
| 1.0 × 10⁵ | 20 | 0 | 0 | 0 | 0 | 0 | 0 | 0 | 0 |
| 0.5 × 10⁶ | 20 | 0 | 0 | 0 | 0 | 4 | 2 | 6 | 30 |
| 1.0 × 10⁶ | 20 | 0 | 0 | 4 | 4 | 3 | 2 | 13 | 65 |
| 0.5 × 10⁷ | 20 | 8 | 6 | 2 | 1 | 1 | 2 | 20 | 100 |
| 1.0 × 10⁷ | 20 | 20 | 0 | 0 | 0 | 0 | 0 | 20 | 100 |

As can be seen from Table 4, no mice in the control group died. No mice died within 5 days in 0.5×10⁵ CFU and 1.0×10⁵ CFU dose groups. The death rate within 5 days in the 0.5×10⁶ CFU dose group was 30%. The death rate within 5 days in the 0.5×10⁷ CFU dose group reached 100%. The death rate within 12 h in the 1.0×10⁷ CFU dose group was 100%. It could be seen that the least fatal dose of C. albicans against the mice was 0.5×10⁷ CFU.

### 2. Challenge Test of Immunized Mice

In the same immunization regimen as that of Example 8, a fatal dose of C. albicans was injected into the tail vein at week 8 for challenge, and the death situation of the mice in each group was observed. The survival rate of the mice was calculated after 7 days, as shown in Table 5.

TABLE 5
Survival Rate of Immunized Mice after Challenge

| | Number | Survival number | Survival |
|---|---|---|---|
| Group | of mice | after 7 days | rate/% |
| Blank group | 20 | 0 | 0 |
| Fluconazole group | 20 | 10 | 50 |
| KLH group | 20 | 6 | 30 |
| Chitosan oligosaccharide group | 20 | 5 | 25 |
| Chitosan oligosaccharide-KLH | 20 | 13 | 65 |
| group (Example 6) | | | |

As can be seen from the above table that the survival rate of mice in the chitosan oligosaccharide-KLH group reached 65%, which was significantly higher than those of other groups, indicating that the prepared oligosaccharide vaccine had an immunoprotective action on infections caused by C. albicans.

It should be finally noted that the above descriptions are merely preferred examples of the present invention, but are not intended to limit the present invention. Although the present invention has been described in detail with reference to the foregoing examples, for a person of ordinary skill in the art, modifications can be still made to the technical solutions described in the foregoing examples, or equivalent replacements can be made to some of the technical solutions. Any modification, equivalent replacement, or improvement made and the like within the spirit and principle of the present invention shall fall within the protection scope of the present invention. The specific implementations of the present invention are described above, but are not intended to limit the protection scope of the present invention. A person skilled in the art should understand that various modifications or deformations may be made without creative efforts based on the technical solutions of the present invention, and such modifications or deformations shall fall within the protection scope of the present invention.

1. A method for preparing an oligosaccharide vaccine for specific prevention of fungal infections, the method comprising:
performing sulfhydrylation modification on a carrier protein containing a primary amino group to obtain a sulfhydrylated protein; and
conjugating the sulfhydrylated protein with a chitosan oligosaccharide mixture and/or chitin oligosaccharide mixture to form the oligosaccharide vaccine.

2. The method for preparing the oligosaccharide vaccine for specific prevention of fungal infections according to claim 1, wherein the chitosan oligosaccharide mixture and/or chitin oligosaccharide mixture have/has a relative molecular weight of ≤5000 Da, and a deacetylation degree of 0-100%.

3. The method for preparing the oligosaccharide vaccine for specific prevention of fungal infections according to claim 1, wherein the carrier protein is a non-humanized protein.

4. The method for preparing the oligosaccharide vaccine for specific prevention of fungal infections according to claim 3, wherein the non-humanized protein is one of Concholepasconcholepas hemocyanin, keyhole limpet hemocyanin, bovine serum albumin, tetanus toxin/toxoid, rotavirus VP7 protein, a diphtheria toxin mutant CRM, and Clostridium perfringens exotoxin/toxoid.

5. The method for preparing the oligosaccharide vaccine for specific prevention of fungal infections according to claim 1, wherein the conjugation is performed in the presence of a bridging agent.

6. The method for preparing the oligosaccharide vaccine for specific prevention of fungal infections according to claim 5, wherein the bridging agent is 3-(2-pyridinedithio) propionohydrazide.

7. The method for preparing the oligosaccharide vaccine for specific prevention of fungal infections according to claim 1, wherein the conjugating the sulfhydrylated protein with the chitosan oligosaccharide mixture and/or chitin oligosaccharide mixture comprises:
adding a bridging agent into a sulfhydrylated protein solution, and performing the reaction at room temperature;
then adding the chitosan oligosaccharide mixture and/or chitin oligosaccharide mixture, and performing the reaction at 4° C.; and
removing small-molecule impurities to obtain the oligosaccharide vaccine.

8. The method for preparing the oligosaccharide vaccine for specific prevention of fungal infections according to claim 1, wherein a molar concentration of the chitosan oligosaccharide mixture and/or chitin oligosaccharide mixture is equivalent to 100 to 1200 times the molar amount of the sulfhydrylated protein; and alternatively the method for removing the small-molecule impurities is dialysis or column chromatography.

9. An oligosaccharide vaccine for specific prevention of fungal infections prepared by the method according to claim 1.

10. A method for treating or preventing systemic fungal infections, or activating Th17 cell immune responses, comprising administering the oligosaccharide vaccine for specific prevention of fungal infections according to claim 9 to a subject.

11. The method for preparing the oligosaccharide vaccine for specific prevention of fungal infections according to claim 2, wherein a molar concentration of the chitosan oligosaccharide mixture and/or chitin oligosaccharide mixture is equivalent to 100 to 1200 times the molar amount of the sulfhydrylated protein; and alternatively the method for removing the small-molecule impurities is dialysis or column chromatography.

12. The method for preparing the oligosaccharide vaccine for specific prevention of fungal infections according to claim 3, wherein a molar concentration of the chitosan oligosaccharide mixture and/or chitin oligosaccharide mixture is equivalent to 100 to 1200 times the molar amount of the sulfhydrylated protein; and alternatively the method for removing the small-molecule impurities is dialysis or column chromatography.

13. The method for preparing the oligosaccharide vaccine for specific prevention of fungal infections according to claim 4, wherein a molar concentration of the chitosan oligosaccharide mixture and/or chitin oligosaccharide mixture is equivalent to 100 to 1200 times the molar amount of the sulfhydrylated protein; and alternatively the method for removing the small-molecule impurities is dialysis or column chromatography.

14. The method for preparing the oligosaccharide vaccine for specific prevention of fungal infections according to claim 5, wherein a molar concentration of the chitosan oligosaccharide mixture and/or chitin oligosaccharide mixture is equivalent to 100 to 1200 times the molar amount of the sulfhydrylated protein; and alternatively the method for removing the small-molecule impurities is dialysis or column chromatography.

15. The method for preparing the oligosaccharide vaccine for specific prevention of fungal infections according to claim 6, wherein a molar concentration of the chitosan oligosaccharide mixture and/or chitin oligosaccharide mixture is equivalent to 100 to 1200 times the molar amount of the sulfhydrylated protein; and alternatively the method for removing the small-molecule impurities is dialysis or column chromatography.

16. The method for preparing the oligosaccharide vaccine for specific prevention of fungal infections according to claim 7, wherein a molar concentration of the chitosan oligosaccharide mixture and/or chitin oligosaccharide mixture is equivalent to 100 to 1200 times the molar amount of the sulfhydrylated protein; and alternatively the method for removing the small-molecule impurities is dialysis or column chromatography.
